(19) United States
(12) Patent Application Publication
(10) Pub. No.: US 2010/0044261 A1
(43) Pub. Date: Feb. 25, 2010

(54) HEALTH MANAGEMENT SUPPLY ORGANIZING SYSTEM AND METHODS

(75) Inventors: Raymond Yao, Ossining, NY (US); Elgin Meike Toepfer, Roesrath (DE); Jan Oliver Seyberth, Chicago, IL (US)

Correspondence Address: DUGAN & DUGAN, P.C., 245 Saw Mill River Road, Suite 309, Hawthorne, NY, 10532, US

(73) Assignee: Bayer HealthCare LLC, Tarrytown, NY (US)

(21) Appl. No.: 12/389,218

(22) Filed: Feb. 19, 2009

Related U.S. Application Data
(60) Provisional application No. 61/090,556, filed on Aug. 20, 2008.

(51) Int. Cl. A61B 19/02

(52) U.S. Cl. 206/370

(57) ABSTRACT

In some aspects, a health monitoring supply organizing system is provided. The supply organizing system includes a large wallet having at least one compartment, and a small wallet received in the compartment for storing and organizing a first subset of the supplies (e.g., diabetes testing and/or control supplies) carried by the system. A user may use the small wallet for short durations (e.g., day trips), while the large wallet may store a remainder of the supplies needed for blood analyte management over a longer period. Numerous other aspects are provided.

### RELATED APPLICATIONS

The present application claims priority to U.S. Provisional Patent Application No. 61/090,556, filed Aug. 20, 2008, and entitled “BLOOD GLUCOSE MONITORING SUPPLY ORGANIZING SYSTEMS” (Attorney Docket No. BHDD-011/L), which is hereby incorporated herein by reference in its entirety for all purposes.

### FIELD OF THE INVENTION

The present invention relates to health management, and more particularly to apparatus for organizing supplies used in health management, such as for blood glucose testing and/or control.

### BACKGROUND OF THE INVENTION

The monitoring of analyte concentration levels in a blood may be an important part of health management (testing and/or control). For example, electrochemical analyte sensors (sometimes referred to as “test strips”) may be used for the monitoring of a patient's blood glucose level as part of diabetes testing. In blood glucose testing, for example, the patient may use a portable lancet device which may be a spring-loaded, trigger-releasable device which receives a single-use, disposable lancet. When the lancet is released, it may prick the user's body part to produce a droplet of blood. That blood droplet may then be transferred to a test strip which interfaces with, and is received within, a port in a blood glucose meter. The test strips may also be single use and disposable. Depending on the meter reading, a user may need to undertake control measures, such as by taking a glucose tablet or insulin. The user may also need to use and carry other testing supplies, such as logbooks (diaries), test strip vials, control or normal solutions, and other testing supplies. Further, the user may need to carry control supplies such as insulin, glucose tablets and insulin delivery devices (e.g., syringes, pens, pen needles), and other control supplies.

It would, therefore, be beneficial to provide a system for containing and organizing such health management supplies to allow for compact storage of, and easy access to, such supplies.

### SUMMARY OF THE INVENTION

In a first aspect, the present invention provides a health supply organizing system, including a large wallet adapted to receive health management supplies, the large wallet including at least one compartment; and a small wallet received in the at least one compartment of the large wallet, the small wallet adapted to receive a first subset of the health management supplies, the first subset including at least an analyte meter.

In another aspect, the present invention provides a blood analyte management supply organizing system, including a large wallet adapted to receive health management supplies for blood analyte management, the large wallet including a first compartment and a second compartment, the second compartment adapted to receive a first portion of an amount of the health management supplies carried by the system; and a small wallet received in the first compartment, the small wallet adapted to receive a second portion of the health management supplies, the second portion including at least an analyte meter.

In another system aspect, the present invention provides a blood glucose management supply organizing system, including a large wallet adapted to receive health management supplies for blood glucose management, the large wallet including a first compartment and a second compartment, the second compartment adapted to receive a first portion of an amount of the health management supplies carried by the system; and a small wallet received in the first compartment, the small wallet having a first leaf and a second leaf foldable about a central portion wherein the small wallet is adapted to receive a second portion of the health management supplies, the second portion including at least an analyte meter.

According to a method aspect, the present invention provides a method of using a health management supply organizing system, including providing a large wallet adapted to receive health management supplies sufficient for health management over a first long period of time, the large wallet including at least one compartment adapted to receive a small wallet, and removing the small wallet containing a first subset of the health management supplies, the first subset including a sufficient amount of the health management supplies for a second shorter period of time as compared to the first long period of time, the first subset including at least an analyte meter.

Other features and aspects of the present invention will become more fully apparent from the following detailed description, the appended claims and the accompanying drawings.

### BRIEF DESCRIPTION OF THE DRAWINGS

FIG. 1 is a top plan view of an exemplary embodiment of a large wallet of a health management supply organizing system provided according to the present invention.

FIG. 2 is a front plan view of the large wallet of FIG. 1.

FIG. 3 is a left side plan view of the large wallet of FIG. 1.

FIG. 4 is a top plan view of the large wallet of FIG. 1 with the flap opened and turned back.

FIG. 5 is a top plan view of an inside of a small wallet which is received in a compartment of a large wallet of a health management supply organizing system provided according to the present invention.

FIG. 6 is a top plan view of the small wallet of FIG. 5, shown closed.

FIG. 7 is a bottom plan view of the small wallet of FIG. 5, shown closed.

FIG. 8 is a top plan view of an inside of another embodiment of a small wallet, shown as opened.

### DETAILED DESCRIPTION

According to a first aspect of the present invention, a health management supply organizing system is provided. An exemplary health management supply organizing system (hereinafter otherwise referred to as a “supply organizing system,” an “organizing system,” or simply a “system”) may include a large wallet adapted to receive testing and/or control supplies for blood management (e.g., blood glucose testing and/or control). The large wallet may include at least one compartment, and a small wallet received in the at least one compartment of the large wallet. The small wallet may be adapted to receive a first subset of the health management supplies including at least an analyte meter. As such, the small wallet may be used for short durations (e.g., day trips) and may carry a minimal amount of testing supplies (e.g., an analyte meter such as a glucose meter and a lancet device, and may further include an insulin delivery device such as an insulin pen and possibly insulin (or tabs)), thus allowing more compact, smaller overall package size. As such, the small wallet is much easier to carry and/or conceal.

The large wallet may be used to store additional health management supplies which may be used to re-supply the small wallet as those supplies are used. Thus, the large wallet may function as a “home base” for storing the additional health management supplies, such as for longer trips or periods of time whereas the small wallet may allow supplies sufficient for shorter excursions. As such, the user may conveniently carry only a subset (less than all) of the health management supplies needed for a shorter time period, and the organizer system may allow for compact storage of such supplies for a longer time period.

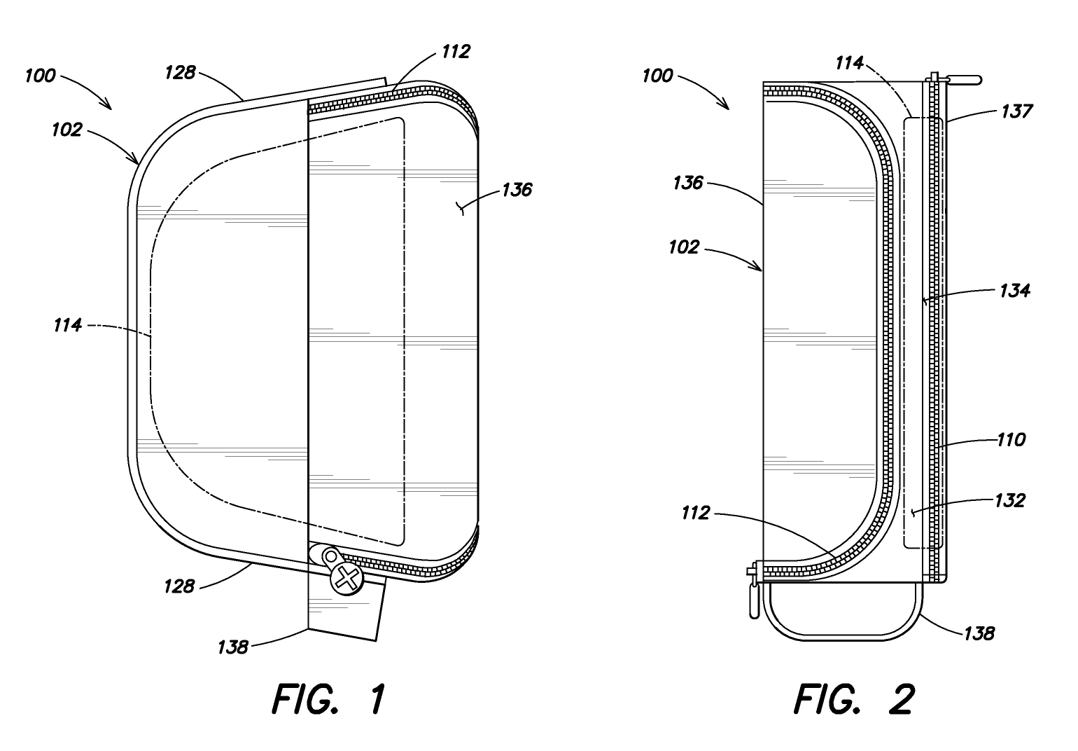

These and other embodiments of the health management supply organizing system of the present invention are described below with reference to FIGS. 1-8.

FIG. 1 is a top plan view of an exemplary embodiment of a health management supply organizing system 100 provided according to the present invention. The organizing system 100 may include a large wallet 102 adapted to receive testing and/or control supplies, such as for analyte monitoring (e.g., blood glucose monitoring). The health management supplies may include an analyte meter. The term “analyte meter,” as used herein, includes a blood analyte meter, an insulin pump, a controller for an insulin delivery system, an insulin pump/glucose meter combination, a patch pump, a continuous glucose meter, or other like metering devices. The health management supplies may also include a lancet device, disposable test strips, disposable lancets, insulin delivery devices (e.g., insulin pens), insulin supplies, vials for storage of test strips, swabs, insulin, glucose tablets, syringes, insertion devices, insulin patches, patch transmitters, cannulas, infusion pump cartridges, adapters, and/or infusion sets for patch pumps, log books, etc. The large wallet may contain a larger amount of the supplies, such as for an extended period of time (e.g., a week or more). The organizing system 100 may be used to organize supplies for monitoring and/or control of a blood analyte, such as glucose, hemoglobin Alc, lipids such as LDL, HDL and triglycerides, lactate, keytone, and other analytes.

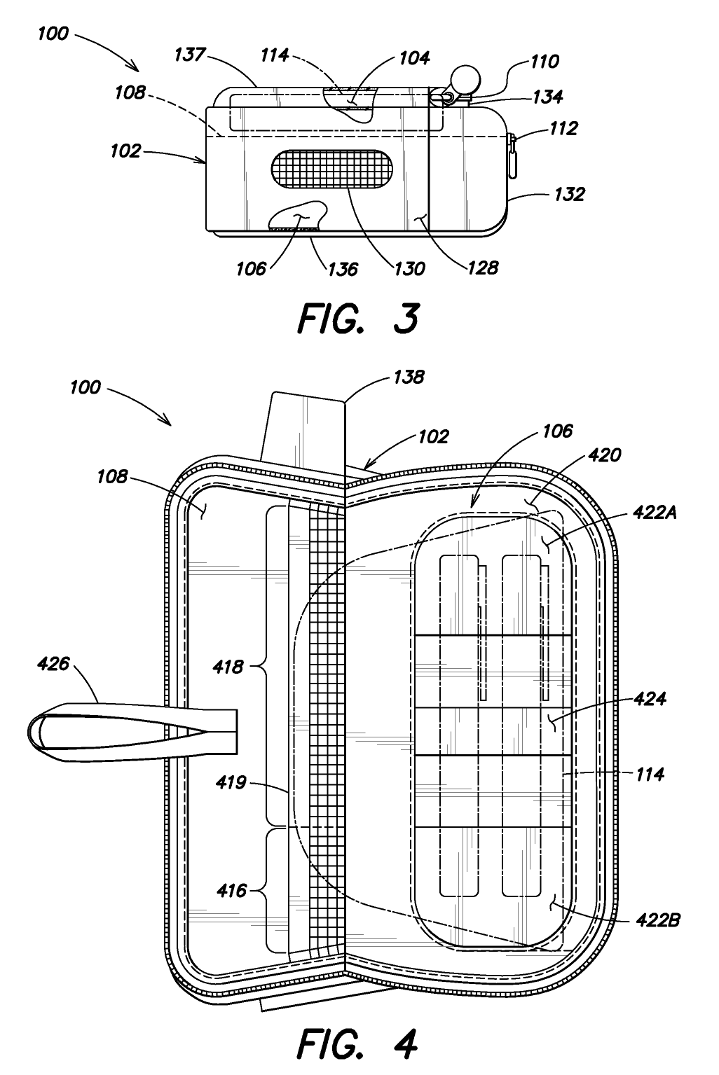

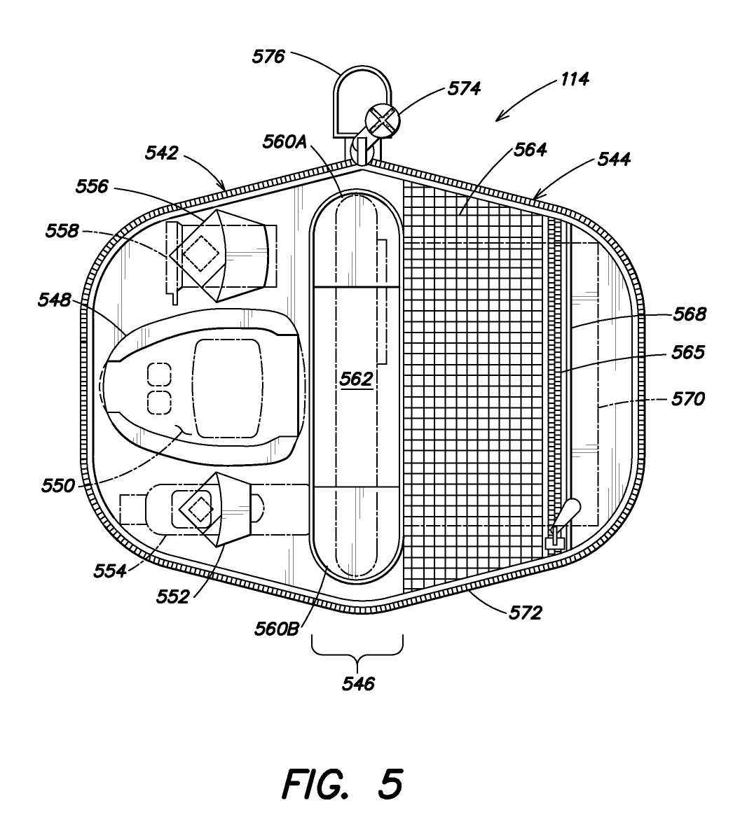

The large wallet 102 may include at least one compartment, and may in some embodiments include at least a first compartment 104 and second compartment 106 (see FIG. 3), which may be located in the bottom and top of the wallet, respectively. The compartments 104, 106 may be separated by an internal partition 108 (shown dotted in FIG. 3) such as a cloth partition, for example. The first and second compartments 104, 106 may be accessed and closed by zippers 110 and 112, for example. Other forms of closure mechanisms may be utilized (e.g., Velcro, snaps, buttons, magnets, etc.). The supply organizing system 100 may also include a small wallet 114. The small wallet 114 (shown dotted in FIGS. 1-3) may be received in the at least one compartment (e.g., the first compartment 104) of the large wallet 102 and is adapted to be removable from the large wallet 102. The small wallet 114, examples of which are illustrated in FIGS. 5-8, may be adapted to receive a subset of the testing and/or control supplies contained in the organizing system 100. The use of the terms “large” and “small” are used herein as relative terms, and do not denote any particular size. The sizes of each of the large and small wallets described herein may be as large or small as needed for the application, albeit the small wallet may be relatively smaller that the large wallet receiving it. In some instances, the small wallet 114 may be folded to reduce its size when stored in the large wallet 102.

Now referring to FIG. 4, the supply organizing system 100 may include, as heretofore described, the small wallet 114 in the first compartment (underneath partition 108 shown), and the second compartment 106 may include one or more pockets 416, 418 adapted to receive testing and/or control supplies. For example, lancets, insulin delivery devices (e.g., insulin pens or syringes), insertion devices, insulin patches, patch transmitters, cannulas, infusion pump cartridges, adapters, and/or infusion sets may be received in the first pocket 416. Other supplies, such as swabs, glucose tabs, insulin supplies, insulin, test strips and/or test strip vials, etc., may be received in a second pocket 418. Additional storage space may be provided above the pockets 416, 418 for quick reference documents or other instructional books, log books, containers for sharps, etc.

In some embodiments, the pockets 416, 418 may be constructed of a mesh material (e.g., a nylon mesh material), and may have a band 419 stitched onto an edge thereof. In some embodiments, the pockets may be insulates or have room for a cool pack so that insulin may be stored therein. Other materials and/or configurations may be used. Pockets 416, 418 may extend between the sides of the large wallet 102. A stitch line may be used to delineate the first pocket 416 from the second pocket 418. Additionally, personal or travel items may be received in the second compartment 106, as well.

An underside of a flap 420 of the large wallet 102 may include one or more elastic members such as elastic pockets 422A, 422B adapted to receive and secure one or more insulin delivery devices (shown dotted), such as insulin pens, and/or syringes. In the depicted embodiment, elastic fabric pockets 422A, 422B and optional strap 424 may be made of a flexible material, such as a flexible or elastic fabric material and may be formed by stitching via thread into a panel forming the flap 420. In some embodiments, the material may be an elastomer material (e.g., an elastomer foam) covered by a fabric sheet on one or both sides. Straight stitch segments provided along a center of each elastic pocket 422A, 422B and the strap 424 separates the pockets and strap into multiple pockets, each of which is adapted to receive an insulin delivery device, for example.

The flap 420 may include a first sheet of material forming the inside panel and a second sheet forming the outside panel. The sheets may be made of the same or different material, for example. In some embodiments, the inside panel may be fluid impermeable (e.g., a polymer sheet) while the outside cover may be a cloth fabric. Various portions of the large wallet 102 may be stiffened by cardboard or other rigid sheet inserts, for example.

The large wallet 102 may further include a hanging loop 426 which may be used to hang the large wallet 102 in a position such that pockets 416 and 418 are oriented with their openings being positioned vertically. The hanging loop 426 may be hung with the flap 420 open, for example, on a coat hanger, peg or hook, allowing easy access to supplies such as during refilling of the small wallet 114. Again referring to FIGS. 1-3, the large wallet 102 may include side walls 128 which may include one or more vents 130. The one or more vents 130 may be formed of a mesh material (e.g., a nylon mesh) stitched into at least one of the side walls. The purpose of the vent 130 is to allow air flow between the one or more compartments and the atmosphere.

As best depicted in FIGS. 2 and 3, the large wallet 102 may include a first frontal surface 132 which may be generally planar and a second frontal surface 134 offset from the first frontal surface 132. The zipper 110 providing access to a first compartment 104 may be formed, at least in part, on the second frontal surface 134. Alternatively, the zipper may wrap around onto the side walls 128 of the large wallet 102 (such as side walls 128) somewhat. Furthermore, the large wallet 102 may include a top surface 136 which may be generally planar and which may be formed generally perpendicular to the first frontal surface 132. The zipper 110 is positioned to form a flap proximate to a bottom 137 of the large wallet 102. The zipper 112 providing access to the second compartment 106 may be formed on the first frontal surface 132 and also the top surface 136, for example. A handle 138 adapted for carrying the system 100 may extend from the top surface 136 to the bottom surface 137 along a side of the large wallet 102.

Turning now to FIGS. 5-7, a first embodiment of a small wallet 114 is illustrated. As before stated, the small wallet 114 is adapted to be received in a compartment (e.g., the first compartment 104) of the large wallet 102 (FIGS. 1-4). The small wallet 114 may include a first leaf 542 and a second leaf 544 which may be foldable about a central portion 546. An inside of the first leaf 542 may include a pocket 548 adapted to receive an analyte meter 540 (e.g., a glucose meter 550. In some embodiments, the pocket 548 may be formed of a flexible plastic material. The material may be opaque, for example. Suitable materials include flexible polymers (e.g., thermoplastic elastomers). Other suitable materials may be used. The pocket 548 may take on an approximate shape of the meter 550 and may be stitched or otherwise fastened along its edges to the first leaf 542. The pocket 548 may include a transparent panel across a front of the meter or may have an opening allowing the meter display to be viewed and any meter buttons to be accessed. In at least one embodiment, a portion of the pocket 548 may be open (e.g., a top, bottom or side) in a region of the meter receiving a test strip. In this manner, the meter 550 may be used for testing without being removed from the pocket 548.

Further, the first leaf 542 may include an elastic strap 552 adapted to receive a lancet device 554. Elastic strap 552 may be doubled over on itself and stitched with a square pattern stitching to the first leaf 542, for example. The strap 552 may be made from any elastic fabric material. Other forms of straps may be used, such as loops of flexible cord material, for example. The lancet device 554 may be an Ascensia® MICROLET® available from Bayer Healthcare LLC of Mishawaka, Ind., for example. Other lancet devices may be employed.

The lancet device 554 may be secured in the small wallet 114 in a position such that it need not be removed to be used. For example, it may be cocked in place and the trigger may be accessible while received in the strap 552. Similarly, the lancet device 554 may be oriented such that a tip including a lancet is directed away from the central portion 546, for example. Positioned on an opposite side of the meter 550 from the lancet device 554 may be another elastic strap 556 similar to the elastic strap 552. This strap 556 is adapted to receive a test strip vial 558. The test strip vial 558 may contain test strips (not shown) for use with the meter 550. The strap 556 and vial 558 may be oriented such that the contents of the vial 558 may be readily accessed without removing the vial 558 from the strap 556.

In the depicted embodiment, the central portion 546 of the wallet 114 may include one or more elastic members (e.g., 560A, 560B) forming pockets or straps adapted to receive and secure an insulin delivery device 562 (e.g., an insulin pen or syringe) onto the wallet 114. In the depicted embodiment, the one or more elastic members 560A, 560B may be pockets formed on opposite ends of the wallet 114. As shown, the device 562 may be secured at one or both of its ends. Alternatively or in addition, pockets may be provided in this area which may be insulated, may include a portion for a cold pack, and may be used for cold storage of insulin.

In some embodiments, the second leaf 544 of the wallet 114 may include a zippered pocket 564 adapted to receive lancets and/or control solution, etc. The pocket 564 may be formed of a mesh fabric, for example, and may include a zipper 565 along an opening spaced from the central portion 546. The pocket 564 may be secured to the second leaf 544 around all its edges, except the edge including the zipper 565. Underneath the zippered pocket 564, an underpocket 568 may be provided. The underpocket 568 may be adapted to receive a logbook 570, for example.

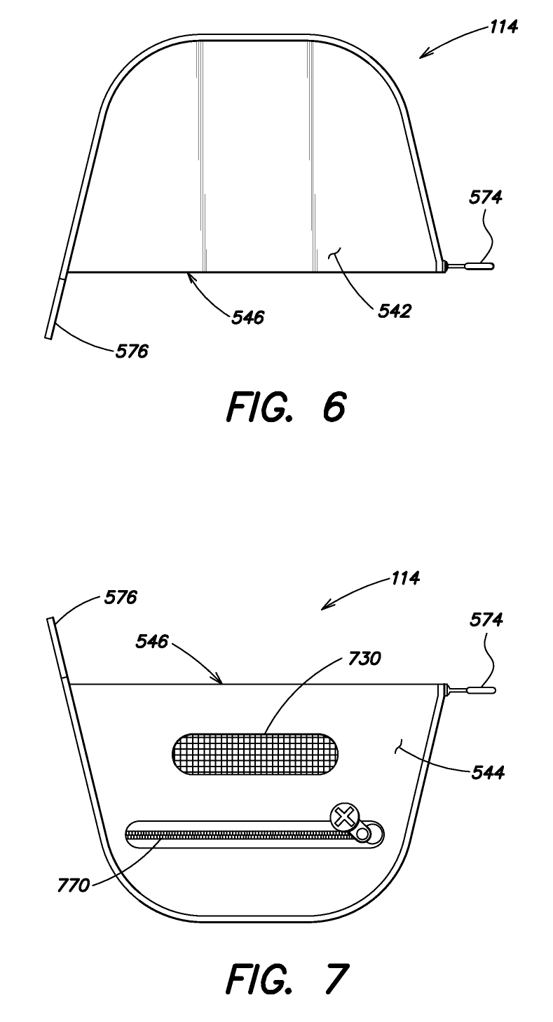

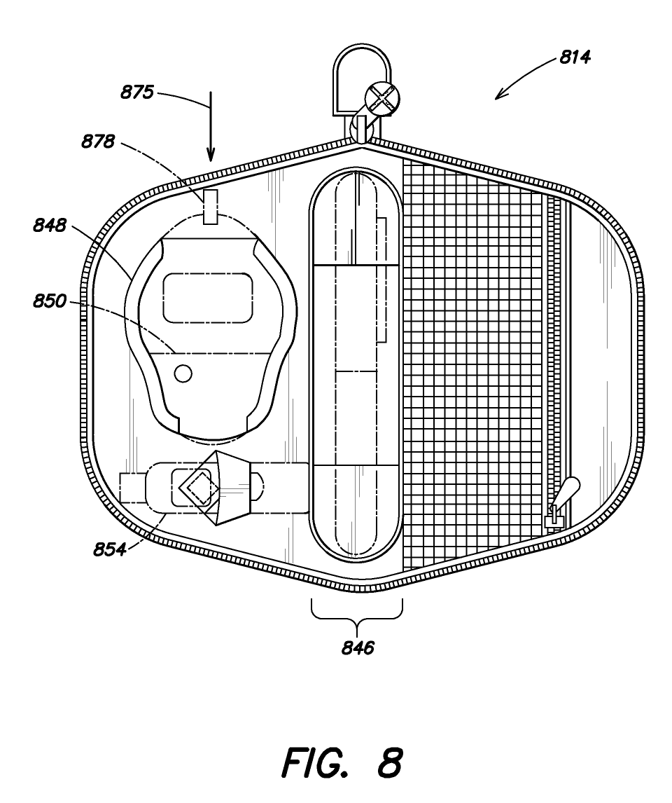

The wallet 114 may include a zipper 572 secured around a periphery of the wallet 114 and edges of the first leaf 542 and second leaf 544. A zipper pull 574 may be provided on the zipper 572 and may include an insignia of the manufacturer, for example. A handle 576 may be provided, such as a tab, strap, carabineer, fastener, or the like for being grasped by the user, or for securing the wallet to the user. As best shown in FIGS. 6 and 7, the first leaf 542 and second leaf 544 may have a generally trapezoidal shape with the central portion 546 being longer than the other sides. Other shapes may be used. The second leaf 544 may further include a vent 730 operative with the inner chamber of the wallet 114 to allow air exchange with the atmosphere. Another pocket 770 may be provided on the leaf 544 to allow access into the underpocket 568, or a separate pocket formed between sheets of material forming the second leaf 544.

FIG. 8 illustrates another embodiment of a small wallet 814. This embodiment is similar to the previous embodiment except that the wallet 814 may be adapted for use with another type of analyte meter. For example, the meter may be a multi-strip meter 850, such as the Ascensia® Breeze® 2 Blood Glucose Meter available from Bayer Healthcare, for example. Other multi-strip analyte meters may be used. Accordingly, because the strips are housed within the meter 850, the strap for the vial included in the previous embodiment may be removed. Additionally, the meter 850 and pocket 848 may be oriented such that the meter 850 is received in a direction parallel (indicated by arrow 875) to an axis running along the central portion 846. As in the previous embodiment, a test strip 878 received in the meter 850 may be accessed without needing to remove the meter 850 from the wallet 814. Similarly, a lancet member 854 may be operated without removing the lancet member 854 from the wallet 814. Any other type of analyte meter, as defined above, may be used with the wallet 814.

In accordance with another aspect of the invention, a method of using a health management supply organizing system 100 of the invention is provided. The method may include, but is not limited to, a step of providing a large wallet adapted to receive health management supplies (testing and/or control supplies) sufficient for management of an analyte in the blood (e.g., blood glucose management) for a first long period of time (e.g., a week or two or more). As described above, the large wallet may include at least one compartment adapted to receive a small wallet of the type described with reference to FIGS. 5-8, for example. In another step, a user removes the small wallet containing a subset of the management supplies (testing and/or control supplies) from the large wallet. The small wallet may include a first subset of the supplies that are carried by the system. The first subset may include a sufficient amount of supplies for a second shorter period of time as compared to the first long period of time. For example, the small wallet may include an amount of supplies for less than a week, for example (e.g., for a day or two, etc.). In particular, the first subset of the supplies provided and carried by the user in the small wallet may include at least an analyte meter, such as blood glucose meter, for example. The first subset may further include a lancet device. In some embodiments, the first subset may include at least a blood glucose meter, a lancet device, and an insulin delivery device.

The foregoing description discloses only exemplary embodiments of the invention. Modifications of the above disclosed supply organizing system and wallets which fall within the scope of the invention will be readily apparent to those of ordinary skill in the art. Accordingly, while the present invention has been disclosed in connection with exemplary embodiments thereof, it should be understood that other embodiments may fall within the spirit and scope of the invention, as defined by the following claims.

The invention claimed is:

1. A health management supply organizing system, comprising:
a large wallet adapted to receive health management supplies, the large wallet including at least one compartment; and
a small wallet received in the at least one compartment of the large wallet, the small wallet adapted to receive a first subset of the health management supplies, the first subset including at least an analyte meter.

2. The health management supply organizing system of claim 1 wherein the large wallet comprises a first compartment and a second compartment.

3. The health management supply organizing system of claim 2 wherein the small wallet is received in the first compartment and the second compartment includes at least one pocket adapted to receive a second subset of the management supplies.

4. The health management supply organizing system of claim 1 wherein the at least one compartment of the large wallet includes a pocket or strap adapted to receive and secure an insulin delivery device.

5. The health management supply organizing system of claim 1 wherein the large wallet comprises side walls and a vent is formed in at least one of the side walls.

6. The health management supply organizing system of claim 1 wherein the large wallet comprises a first frontal surface and a second frontal surface offset from the first frontal surface and a zipper providing access to a first compartment is formed at least in part on the second frontal surface.

7. The health management supply organizing system of claim 1 wherein the large wallet comprises a first fontal surface and a top surface generally perpendicular to the first frontal surface and a zipper providing access to a second compartment is formed on the first frontal surface and the top surface.

8. The health management supply organizing system of claim 1 wherein the small wallet comprises a first leaf and a second leaf foldable about a central portion.

9. The health management supply organizing system of claim 8 wherein the first leaf includes a pocket adapted to receive the analyte meter.

10. The health management supply organizing system of claim 8 wherein the first leaf includes an elastic strap adapted to receive and secure a lancet device.

11. The health management supply organizing system of claim 8 wherein the first leaf includes an elastic strap adapted to receive and secure a test strip vial.

12. The health management supply organizing system of claim 8 wherein the central portion includes an elastic member adapted to receive and secure an insulin delivery device.

13. The health management supply organizing system of claim 8 wherein the second leaf includes a zippered pocket.

14. The health management supply organizing system of claim 13 wherein the second leaf includes a pocket formed under the zippered pocket which is adapted to receive a logbook.

15. The health management supply organizing system of claim 8 wherein the first leaf and second leaf have a generally trapezoidal shape.

16. A blood analyte management supply organizing system, comprising:
a large wallet adapted to receive health management supplies for blood analyte management, the large wallet including a first compartment and a second compartment, the second compartment adapted to receive a first portion of an amount of the health management supplies carried by the system; and
a small wallet received in the first compartment, the small wallet adapted to receive a second portion of the health management supplies, the second portion including at least an analyte meter.

17. A blood glucose management supply organizing system, comprising:
a large wallet adapted to receive health management supplies for blood glucose management, the large wallet including a first compartment and a second compartment, the second compartment adapted to receive a first portion of an amount of the health management supplies carried by the system; and
a small wallet received in the first compartment, the small wallet having a first leaf and a second leaf foldable about a central portion wherein the small wallet is adapted to receive a second portion of the health management supplies, the second portion including at least an analyte meter.

18. A method of using a health management supply organizing system, comprising:
providing a large wallet adapted to receive health management supplies sufficient for health management over a first long period of time, the large wallet including at least one compartment adapted to receive a small wallet, and
removing the small wallet containing a first subset of the health management supplies, the first subset including a sufficient amount of the health management supplies for a second shorter period of time as compared to the first long period of time, the first subset including at least an analyte meter.

19. A method of using a health management supply organizing system of claim 18 wherein the first subset includes at least the analyte meter and a lancet device.

20. A method of using a health management supply organizing system of claim 18 wherein the first subset includes at least a blood glucose meter, a lancet device, and an insulin delivery device.
